(19) United States
(12) Patent Application Publication
(10) Pub. No.: US 2008/0009051 A1
(43) Pub. Date: Jan. 10, 2008

(54) Method and Device for Isolating Cells

(75) Inventors: Assaf Deutsch, Moshav-Tzfaria (IL); Mordechai Deutsch, Moshav Olesh Doar-Na Lev HaSharon (IL)

Correspondence Address: Martin D. Moynihan;PRTSI, P.O.Box 16446, Arlington, VA, 22215, US

(73) Assignee: Seng Enterprises Ltd., Larnaca (CY)

(21) Appl. No.: 11/660,783

(22) PCT Filed: Aug. 24, 2005
(86) PCT No.: PCT/IL05/00914

§ 371 Date: Feb. 22, 2007

Related U.S. Application Data
(60) Provisional application No. 60/604,010, filed on Aug. 25, 2004.

(51) Int. Cl. C12N 13/00; C12M 1/36

(52) U.S. Cl. 435/173.9; 435/286.2

(57) ABSTRACT

A method for selecting specific cells from amongst a group of cells and a device for implementing the method, the method including the use of a radiation sensitive medium having a first state that does not immobilize cells and a second state that immobilizes cells wherein the radiation sensitive medium locally transforms from the first state to the second state upon exposure to radiation.

### FIELD AND BACKGROUND OF THE INVENTION

The present invention relates to the field of cellular biology and more particularly, to a method and device for isolating living cells. Specifically, the present invention is of a method and a device that allows for the isolation of living cells having desired attributes, characteristics or properties from a group of cells by immobilizing cells not having the desired attributes, characteristics or properties.

Combinatorial methods in chemistry, cellular biology and biochemistry are essential for the near simultaneous preparation of multitudes of active entities such as molecules. Once such a multitude of molecules is prepared, it is necessary to study the effect of each one of the active entities on a living organism.

The study of the effects of stimuli such as exposure to active entities on living organisms is preferably initially performed on living cells. Since cell-functions include many interrelated pathways, cycles and chemical reactions, the study of an aggregate of cells, whether a homogenous or a heterogeneous aggregate, does not provide sufficiently detailed or interpretable results: rather a comprehensive study of the biological activity of an active entity may be advantageously performed by examining the effect of the active entity on a single isolated living cells. Thus, the use of single-cell assays is one of the most important tools for understanding biological systems and the influence thereupon of various stimuli such as exposure to active entities.

The combinatorial preparation of a multitudes of active entities coupled with the necessity of studying the effect of each one of the active entities on living organisms using single-cell assays, requires the development of high-throughput single live cell assays. There is a need for the study of real-time responses to stimuli in large and heterogeneous cell populations at an individual cell level. In such studies it is essential to have the ability to define multiple characteristics of each individual cell, as well as the individual cell response to the experimental stimulus of interest.

In the art, various different methods for studying living cells are known.

Multiwell plates having 6, 12, 48, 96, 384 or even 1536 wells on a standard ca. 8.5 cm by ca. 12.5 cm footprint are well known in the art. Such multiwell plates are provided with an 2 n by 3 n array of rectangular packed wells, n being an integer. The diameter of the wells of a plate depends on the number of wells and is generally greater than about 250 microns (for a 1536 well plate). The volume of the wells depends on the number of wells and the depth thereof but generally is greater than 5×10⁻⁶ liter (for a 1536 well plate). The standardization of the multiwell plate format is a great advantage for researchers, allowing the use of standardized products including robotic handling devices, automated sample handlers, sample dispensers, plate readers, observation components, plate washers, software and such accessories as multifilters.

Multiwell plates are commercially available from many different suppliers. Multiwell plates made from many different materials are available, including but not limited to glass, plastics, quartz and silicon. Multiwell plates having wells where the inside surface is coated with various materials, such as active entities, are known.

Although exceptionally useful for the study of large groups of cells, multiwell plates are not suitable for the study of individual cells or even small groups of cells due to the large, relative to the cellular scale, size of the wells. Cells held in such wells either float about a solution or adhere to a well surface. When cells float about in a well, specific individual cells are not easily found for observation. When cells adhere to a well surface, the cells adhere to any location in the well, including anywhere on the bottom and walls of the well. Such variability in location makes high-throughput imaging (for example for morphological studies) challenging as acquiring an individual cell and focusing thereon is extremely difficult. Such variability in location also makes high-throughput signal processing (for example, detection of light emitted by a single cell through fluorescent processes) challenging as light must be gathered from the entire area of the well, decreasing the signal to noise ratio. Further, a cell held in a well of a multiwell plate well can be physically or chemically manipulated (for example, isolation or movement of a single selected cell or single type of cell, changing media or introducing active entities) only with difficulty. Further, the loading of multiwell plates when loaded with one cell per well, expressed in terms of cells held singly in the wells per unit area is very low (about 1536 cells in 65 cm², or 24 cells cm⁻²). Thus, multiwell plates are in general only suitable for the study of homogenous or heterogenous aggregates of cells as a group.

Multiwell plates are unsuitable for the study of cells undergoing apoptosis. It is known to study biological processes by exposing a monolayer of cells adhering to the bottom of the well of a multiwell plate to a stimulus that causes apoptosis. However, once a cell begins the apoptosis process, the adhesion of the cell to the bottom of the well is no longer sufficient: the cell detaches from the bottom and is carried away by incidental fluid currents in the well. The cell is no longer observable and its identity lost.

Multiwell plates are also unsuitable for the study of non-adhering cells. Just as cells undergoing apoptosis, in multiwell plates non-adhering cells can be studied as individuals only with difficulty. This is a major disadvantage as non-adhering cells are crucial for research in drug discovery, stem cell therapy, cancer and immunological diseases detection, diagnosis and therapy. For example, blood contains seven heterogeneous types of non-adherent cells, all which perform essential functions, from carrying oxygen to providing immunity against disease.

In the art, a number of method and devices have been developed for the study of individual cells or a small number of cells as a group. Many such methods are based on using picowell-bearing device. A picowell-bearing device is a device for the study of cells that has at least one picowell-bearing component for study of cells. A picowell-bearing component is a component having at least one, but generally a plurality of picowells, each picowell configured to hold at least one cell. The term “picowell” is general and includes such features as dimples, depressions, tubes and enclosures. Since cells range in size from about 1 microns to about 100 (or even more) microns diameter there is no single picowell size that is appropriate for holding a single cell of any type. That said, the dimensions of the typical individual picowell in the picowell-bearing components known in the art have dimensions of between about 1 microns up to about 200 microns, depending on the exact implementation. For example, a device designed for the study of single isolated 20 micron cells typically has picowells of dimensions of about 20 microns. In other cases, larger picowells are used to study the interactions of a few cells held together in one picowell. For example, a 200 micron picowell is recognized as being useful for the study of the interactions of two or three cells, see PCT Patent Application No. IL01/00992 published as WO 03/035824.

One feature that increases the utility of a picowell-bearing device is that each individual picowell is individually addressable. By individual addressability is meant that each picowell can be registered, found, observed or studied without continuous observation. For example, while cells are held in picowells of a picowell-bearing component, each cell is characterized and the respective picowell wherein that cell is held is noted. When desired, the observation component of the picowell-bearing device is directed to the location of the picowell where a specific cell is held. One method of implementing individual addressability is by the use of fiducial points or other features (such as signs or labels), generally on the picowell-bearing component. Another method of implementing individual addressability is by arranging the picowells in a picowell-array and finding a specific desired picowell by counting. Another method of implementing individual addressability is by providing a dedicated observation component for each picowell.

In the art, the picowell-bearing component of a picowell-bearing device is often a chip, a plate or other substantially planar component. Herein such a component is termed a “carrier”. In the art, there also exist non-carrier picowell-bearing components of picowell-bearing devices, for example, bundles of fibers or bundles of tubes.

Mrksich and Whitesides, Ann. Rev. Biophys. Biomol. Struct. 1996, 25, 55-78; Craighead et al., J. Vac. Sci. Technol. 1982, 20, 316; Singhvi et al., Science 1994, 264, 696-698; Aplin and Hughes, Analyt. Biochem. 1981, 113, 144-148 and U.S. Pat. No. 5,324,591 all teach devices including arrays of spots of cell-attracting or cell-binding entities on a plate. In such devices, the spots serve as picowells, binding cells through a variety of chemical bonds. In such devices, the plate is the picowell-bearing component of the device. Due to the size of the spots, each such picowell generally holds more than one cell. To reduce interaction between cells held at different picowells, the spots must be spaced relatively far apart, reducing loading as expressed in terms of picowells per unit area. Even with generous spacing, in such picowell-bearing components held cells are not entirely isolated from mutual interaction, nor can cells be subject to individual manipulation. The fact that the cells are not free-floating but are bound to the plate through some interaction necessarily compromises the results of experiments performed.

In U.S. Pat. No. 6,103,479 the picowell-bearing component is a transparent carrier provided with a non-uniform array of picowells, each well functionalized with chemical entities that bind to cells specifically or non-specifically. Each picowell is of approximately 200 to 1000 micron diameter and is configured to hold a plurality of cells. The inter picowell areas are hydrophobic so as not to attract cells. In addition to the carrier, a device of U.S. Pat. No. 6,103,479 is provided with a glass, plastic or silicon chamber-bearing plate in which individually addressable microfluidic channels are etched, the chamber-bearing plate configured to mate with the carrier. When mated, the carrier and chamber-bearing plate constitute a cassette in which each cell is bound to the carrier and isolated in a chamber provided with an individual fluid delivery system. Reagents are provided through the fluid delivery system and observed by the detection of fluorescence. In order to provide space for the walls of the chambers, the inter picowell areas of the carrier are relatively large, reducing loading as expressed in terms of picowells per unit area. Subsequent to study, the cassette is separated into the two parts and the micro-patterned array of cells processed further. In some embodiments, the chamber-bearing plate is made of polytetrafluoroethylene, polydimethylsiloxane or an elastomer. As held cells do not make contact with the chamber-bearing plate it is not clear what advantages are to be had when providing a chamber-bearing plate of such esoteric materials.

In U.S. patent application Ser. No. 10/199,341 a device is taught for trapping a plurality of dielectric objects (such as cells), each individual object in an individual light beam produced by an optical array.

In U.S. Pat. No. 4,729,949 a device is taught for trapping individual cells in a picowell-bearing carrier, the carrier being substantially a plate having a plurality of picowells that are individually-addressable tapered apertures of a size to hold individual cells. Suction applied from the bottom surface of the plate where the picowells are narrow creates a force that draws cells suspended in a fluid above the carrier into the wide end of the picowells on the surface of the carrier to be held therein. Using the teachings of U.S. Pat. No. 4,729,949 a specific group of cells (having dimensions similar to that of the wide end of the picowells) are selected from amongst a group of cells and held in the carrier. Although the cells are subjected to common stimuli, the fact that the picowells are individually addressable allows the effect of a stimulus on an individual cell to be observed. A carrier of U.S. Pat. No. 4,729,949 is generally made of metal such as nickel and prepared using standard photoresist and electroplating techniques. In a carrier of U.S. Pat. No. 4,729,949 the inter picowell areas of the carrier are relatively large, leading to a low loading as expressed in terms of picowells per unit area. Further, the suction required to hold cells in picowells of a carrier of U.S. Pat. No. 4,729,949 deforms held cells and makes a significant portion of the cell membranes unavailable for contact, both factors that potentially compromise experimental results. Study of cells held in a carrier of U.S. Pat. No. 4,729,949 with methods not based on detecting fluoresence generally gives poor results due to reflections of light from the carrier.

In PCT Patent Application No. US99/04473 published as WO 99/45357 is taught a picowell-bearing component produced by etching the ends of a bundle of optical fibers (apparently of glass) while leaving the cladding intact to form a picowell-bearing component that is a bundle of tubes. The size of the hexagonal picowells is demonstrated to be as small as 7 micron wide, 5 micron deep and having a volume of about 1.45×10⁻¹³ liter. The inter picowell area is quite large due to the thickness of the cladding of the optical fibers. Light emitted by cells held in each picowell are independently observable through a respective optical fiber. In some embodiments, the inside surface of the picowells is coated with a film of materials such as collagen, fibronectin, polylysine, polyethylene glycol, polystyrene, fluorophores, chromophores, dyes or a metal. Loading the picowell-bearing component of PCT Patent Application No. US99/04473 includes dipping the optical fiber bundle in a cell suspension so that cells adhere to the picowells. There are a number of disadvantages to the teachings of PCT Patent Application No. US99/04473. The fact that the cells are studied only subsequent to adhesion to the picowells necessarily influences the results of experiments performed. Since cell proliferation generally begins soon after adhesion, it is not known if a detected signal is produced by a single cell or a plurality of cells. It is is not clear where exactly in a picowell a cell is held and therefore what percentage of light emitted from a cell travels to a detector. The fact that emitted light travels through an optical fiber leads to loss of time-dependent and phase information.

In PCT Patent Application No. IL04/00194 of the Applicant filed 26 Feb. 2004 and published as WO 2004/077009 is taught a picowell-bearing component produced by bundling together glass capillaries, each glass capillary attached to an independent fluid flow generator such as a pump. A cell held in a first picowell is transferred to a second picowell by the simultaneous application of an outwards flow from the first picowell and an inwards flow into the second picowell.

A preferred device for the study of cells is described in PCT Patent Application No. IL01/00992 published as WO 03/035824 of the Applicant. The device 10, depicted in FIG. 1, is provided with a transparent carrier 12 as a picowell-bearing component. Carrier 12 is substantially a sheet of transparent material (such as glass or polystyrene) on the surface of which features such as inlet connectors 14, fluid channels 16, picowells (in FIG. 1, a picowell-array 18), a fluid reservoir 20 and an outlet connector 22. Carrier 12 is immovably held in a holder 24 having a cutout window of a size and shape to accept carrier 12. Other components of device 10 not depicted include flow generators, observation components, external tubing and the like. When a cover slip (not depicted) is placed or integrally formed with carrier 12, fluid channels 16, picowell-array 18 and reservoir 20 are sealed forming channels that allow transport of fluids and reagents to cells held in picowell-array 18. The picowells are configured to hold a predetermined number of cells (one or more) of a certain size and are preferably individually addressable both for examination and manipulation.

FIG. 2 is a reproduction of a photograph of a different carrier 26 held in a holder 24. A first syringe 28 as an inlet flow generator is in communication with an inlet connector 14 by a capillary tube 30. Inlet connector 14 is in communication with picowell-array 18 through a fluid passage 16. Picowell-array 18 is in communication with outlet connector 22 through a fluid passage 16. A second syringe 32 as an outlet flow generator is in communication with outlet connector 22 through capillary tube 34.

PCT Patent Application No. IL01/00992 also teaches methods of physically manipulating cells held in a picowell-bearing device using, for example, individually addressable microelectrodes (found in the picowells or in the cover slip) or optical tweezers. Typical physical manipulations include moving selected cells into or out of specific picowells. One useful method that is implemented using a device of PCT Patent Application No. IL01/00992 is that cells, each held alone in a respective picowell, are examined (either in the presence or absence of reagents) and based on the results of the examination, cells with a certain characteristic are selected to remain in a respective picowell while cells without the certain characteristic are removed from a respective picowell and ejected by the application of a flow in parallel to the surface of the carrier, generated by a flow generator.

An additional feature of the teachings of PCT Patent Application No. IL01/00992 is that, in some embodiments, the picowells are juxtaposed, that is, the area occupied by a picowell-array is substantially entirely made up of picowells with little or no inter picowell area, see FIG. 3. FIG. 3 is a reproduction of a photograph of part of a picowell-array 18 from the top of a carrier 12 of PCT Patent Application No. IL01/00992. In FIG. 3 is seen a plurality of hexagonal picowells 36, some populated with living cells 38. It is seen that the inter picowell areas 40 make up only a minor percentage of the total area of picowell-array 18. This feature allows near tissue-density packing of cells, especially in single-cell picowell configurations. For example, a typical device of PCT Patent Application No. IL01/00992 having a 2 mm by 2 mm picowell-array of hexagonally-packed juxtaposed picowells of 10 micron diameter and no inter picowell area includes about 62000 picowells while a device having a 2 mm by 2 mm picowell-array of hexagonally-packed juxtaposed picowells of 20 micron diameter and no inter picowell area includes about 10000 picowells. This feature also allows simple picowell loading: a fluid containing suspended cells is introduced in the volume above the picowell array. Since there is little inter picowell area, cells settle in the picowells.

Using devices such as described above, the properties of individual cells can be studied with great accuracy, either in a natural state or in response to stimuli and individual cells having particular properties can be identified within a population of cells, as detailed for example in U.S. patent application Ser. No. 10/276,080 of the Applicant.

Although the study of cells and identification of various properties is of great scientific and diagnostic interest, it is often desired to isolate cells having certain properties from amongst a population of cells.

As discussed above, in PCT Patent Application No. IL01/00992 of the Applicant is taught the physical manipulation of cells by the use of a picowell-array bearing device having individually adressable microelectrodes associated with individually adressable picowells. A cell held in a picowell is observed and if desired, ejected from the picowell by a force generated using the associated microelectrode. Fluid flowing in parallel to the picowell array carries the cell away, while leaving non-ejected cells in the picowells.

As discussed above, in PCT Patent Application No. IL04/00194 of the Applicant is taught the physical manipulation of cells by the use of a picowell-array bearing device having individually adressable flow generators associated with individually adressable picowells. A cell held in a picowell is observed and if desired, ejected from the picowell by the application of an outwards flow using the associated flow generator and drawn into an adjacent picowell by the application of an inwards flow of the respective flow generator. Preferably, the cell is transferred from one picowell to another until it is ejected or isolated as desired.

A disadvantage of both the above methods is the requirement for each picowell to have an addressable force generator for each picowell, either a microelectrode as in PCT Patent Application No. IL01/00992 or a fluid flow generator as in PCT Patent Application No. IL04/00194.

In PCT Patent Application No. IL01/00992 of the Applicant is also taught the physical manipulation of a cell held in a picowell of a picowell array by the use of optical tweezers. The optical tweezers can only be used serially, rendering isolation of selected cells from a group of cells impractical.

It would be highly advantageous to have a method to isolate cells having specific observed attributes, characteristics or attributes for further study or use from a population of cells not having at least some of the disadvantages of the prior art.

### SUMMARY OF THE INVENTION

The present invention successfully addresses at least some of the shortcomings of the prior art by providing a method of immobilizing at least one selected cell of a group of cells. Specifically, implementation of the method of the present invention allows a large group of cells to be divided into two groups, cells having a desired attribute, property or characteristic and cells devoid of the desired attribute, property or characteristic and to immobilize cells belonging to one of the two groups. One skilled in the art recognizes that such a method has many uses in the biological, pharmaceutical and medical sciences. In a preferred embodiment, the immobilized cells are discarded and/or the non-immobilized cells are isolated.

Thus, according to the teachings of the present invention there is provided a method of immobilizing at least one cell from a plurality of cells comprising: (a) placing the plurality of cells in functional proximity of a radiation sensitive medium, the radiation sensitive medium having a first state that does not immobilize cells and a second state that immobilizes cells wherein the radiation sensitive medium locally transforms from the first state to the second state upon exposure to radiation; (b) selecting at least one cell to be immobilized; and (c) using radiation produced by a radiation source to irradiate the radiation sensitive medium in the first state in the vicinity of the at least one selected cell so as to transform the radiation sensitive medium only in the vicinity of the at least one selected cell to the second state so as to immobilize the at least one selected cell.

In an embodiment of the present invention, the method further comprises (d) subsequent to the irradiation, isolating non-immobilized cells.

In an embodiment of the present invention, the method further comprises (e) subsequent to the irradiation, discarding immobilized cells.

In an embodiment of the present invention, selecting the at least one cell to be immobilized includes: (i) observing a cell from the group of cells; and (ii) designating the observed cell as the selected cell. In an embodiment of the present invention, by observing a cell is meant optical study of the cell. In an embodiment of the present invention, the designation of a cell as a selected cell is contingent on observing a specific property of the observed cell, for example, observing an observable effect indicative of the specific property induced by exposing the group of cells to a stimulus (e.g., exposure to an active entity such as a probe). Thus, in embodiments of the present invention, prior to observing a cell, the cell or cells are exposed to a stimulus, for example to an active entity, for example to a probe.

In an embodiment of the present invention, a selected cell is irradiated prior to observing and designating of a different cell of the group of cells, that is cells are designated and irradiated for immobilization serially.

In an embodiment of the present invention, a plurality of cells is observed and designated as selected cells prior to irradiation, that is, there is a distinct designation step and a distinct irradiation step. Preferably the cells are designated serially or simultaneously but all cells to be immobilized are irradiated simultaneously.

In an embodiment of the present invention, individual cells of the group of cells are arranged substantially coplanarly in the cell holder.

In an embodiment of the present invention, individual cells of the group of cells are arranged in an array.

In an embodiment of the present invention, individual cells of the group of cells are individually addressable.

In an embodiment of the present invention, individual cells of the group of cells are held in picowells, preferably picowells of a picowell array.

In an embodiment of the present invention, the individually addressable cells are associated with individually addressable radiation sources to irradiate the radiation sensitive medium.

In an embodiment of the present invention, during the irradiation a single radiation source is used to sequentially irradiate the vicinity of a plurality of selected cells.

In an embodiment of the present invention, during the irradiation a single radiation source is used to simultaneously irradiate the vicinity of a plurality of selected cells.

In an embodiment of the present invention, cells of the plurality of cells are entirely surrounded by the radiation sensitive medium in the first state. In an embodiment of the present invention, cells of the plurality of cells are not entirely surrounded by the radiation sensitive medium in the first state. In an embodiment of the present invention, cells of the plurality of cells rest on top of the radiation sensitive medium in the first state.

In an embodiment of the present invention, the radiation sensitive medium is more viscous in the second state than in the first state. In an embodiment of the present invention, in the first state the radiation sensitive medium is fluid and in the second state the radiation sensitive medium is a gel. In an embodiment of the present invention, in the first state the radiation sensitive medium is fluid and in the second state the radiation sensitive medium is a solid.

In an embodiment of the present invention, the radiation sensitive medium is more viscous in the first state than in the second state. In an embodiment of the present invention, the second state the radiation sensitive medium is fluid and in the first state the radiation sensitive medium is a gel. In an embodiment of the present invention, in the second state the radiation sensitive medium is fluid and in the first state the radiation sensitive medium is a solid.

In an embodiment of the present invention, the radiation sensitive medium comprises chemical functional groups configured to bind to cells held in the cell holder, wherein in the first state the chemical functional groups are non-reactive and transformation of the radiation sensitive medium to the second state includes radiation-induced activation of the chemical functional groups.

In an embodiment of the present invention, the radiation comprises frequencies selected from the group consisting of visible light, infrared radiation, ultraviolet radiation and microwave radiation. In an embodiment of the present invention, the radiation is collimated. In an embodiment of the present invention, the radiation is coherent. In an embodiment of the present invention, the radiation is substantially monochromatic. In an embodiment of the present invention, the radiation source comprises a laser.

In an embodiment of the present invention the radiation is in the form of a beam. In an embodiment of the present invention the beam has a diameter of less than 50 micron, less than 20 micron, less than 10 micron, less than 5 micron and even less than 2 micron.

According to the teachings of the present invention there is provided a device for immobilizing cells comprising: (a) a cell holder for holding cells having an upper surface; (b) on the upper surface a radiation sensitive medium, the radiation sensitive medium having a first state that does not immobilize cells held in the cell holder and a second state that immobilizes cells held in the cell holder wherein the radiation sensitive medium locally transforms from the first state to the second state upon exposure to radiation; (c) an observation component configured to allow observation of cells held in the cell holder; (d) an irradiation component configured to selectively irradiate the radiation sensitive medium in proximity of selected cells held in the cell holder with radiation; and (e) a control component functionally associated with the irradiation component, configured to direct the irradiation component to irradiate the radiation sensitive medium in proximity of selected cells held in the cell holder.

In an embodiment of the present invention, the observation component is configured to observe cells held in the cell holder from above the upper surface. In an embodiment of the present invention, the observation component is configured to observe cells held in the cell holder from below the upper surface.

In an embodiment of the present invention, the control component is functionally associated with the observation component and configured to autonomously designate cells held in the cell holder as selected cells based on input from the observation component.

In an embodiment of the present invention, the irradiation component is configured to irradiate the radiation sensitive medium from above the upper surface. In an embodiment of the present invention, the irradiation component is configured to irradiate the radiation sensitive medium from below the upper surface.

In an embodiment of the present invention, the radiation comprises frequencies selected from the group consisting of visible light, infrared radiation, ultraviolet radiation and microwave radiation. In an embodiment of the present invention, the radiation is collimated. In an embodiment of the present invention, the radiation is coherent. In an embodiment of the present invention, the radiation is substantially monochromatic. In an embodiment of the present invention, the radiation source comprises a laser.

In an embodiment of the present invention, the irradiation component is configured to illuminate the radiation sensitive medium in proximity of a single cell of a plurality of cells held in the cell holder at any one time.

In an embodiment of the present invention, the irradiation component is configured to project a beam of radiation. In an embodiment of the present invention the beam of radiation has a diameter of less than 50 micron, less than 20 micron, less than 10 micron, less than 5 micron and even less than 2 micron. In an embodiment of the present invention, the beam of radiation is projected substantially coaxially with an optical axis of the observation component

In an embodiment of the present invention, the irradiation component is configured to simultaneously irradiate the vicinity of a plurality of selected cells from a plurality of cells held in the cell holder at any one time.

In an embodiment of the present invention, the irradiation component comprises a plurality of independently addressable radiation sources.

In an embodiment of the present invention, the control component comprises an adjustable mask. In an embodiment, an adjustable mask is a device optically interposed between the illumination source and the upper surface, and configured to have a changing pattern of transparent areas and opaque areas, the transparent areas allowing radiation to pass through the adjustable mask and illuminate the upper surface and the opaque areas preventing radiation from passing through the adjustable mask.

In an embodiment of the present invention, in the first state, the radiation sensitive medium is configured to entirely surround cells held cells held in the cell holder. In an embodiment of the present invention, in the first state, the radiation sensitive medium is configured to only partially surround cells held cells held in the cell holder so that at least part of a the surrounded cell emerges from the radiation sensitive medium. In an embodiment of the present invention, in the first state, the radiation sensitive medium is configured to allow cells held cells held in the cell holder to rest thereupon.

In an embodiment of the present invention, the radiation sensitive medium is more viscous in the second state than in the first state. In an embodiment of the present invention, in the first state the radiation sensitive medium is fluid and in the second state the radiation sensitive medium is a gel. In an embodiment of the present invention, in the first state the radiation sensitive medium is fluid and in the second state the radiation sensitive medium is a solid.

In an embodiment of the present invention, the radiation sensitive medium is more viscous in the first state than in the second state. In an embodiment of the present invention, the second state the radiation sensitive medium is fluid and in the first state the radiation sensitive medium is a gel. In an embodiment of the present invention, in the second state the radiation sensitive medium is fluid and in the first state the radiation sensitive medium is a solid.

In an embodiment of the present invention, the radiation sensitive medium comprises chemical functional groups configured to bind to cells held in the cell holder, wherein in the first state the chemical functional groups are non-reactive and transformation of the radiation sensitive medium to the second state includes radiation-induced activation of the chemical functional groups.

In an embodiment of the present invention, the upper surface is substantially smooth.

In an embodiment of the present invention, the device further comprises localizing features (e.g., picowells) on the upper surface configured to localize cells held in the cell holder at specific locations of the upper surface.

In an embodiment of the present invention, the device further comprises at least one active entity dispenser, configured to dispense an active entity onto the upper surface.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 (prior art) depicts a picowell-bearing device for the study of cells of PCT Patent Application No. IL01/00992 including a transparent carrier;

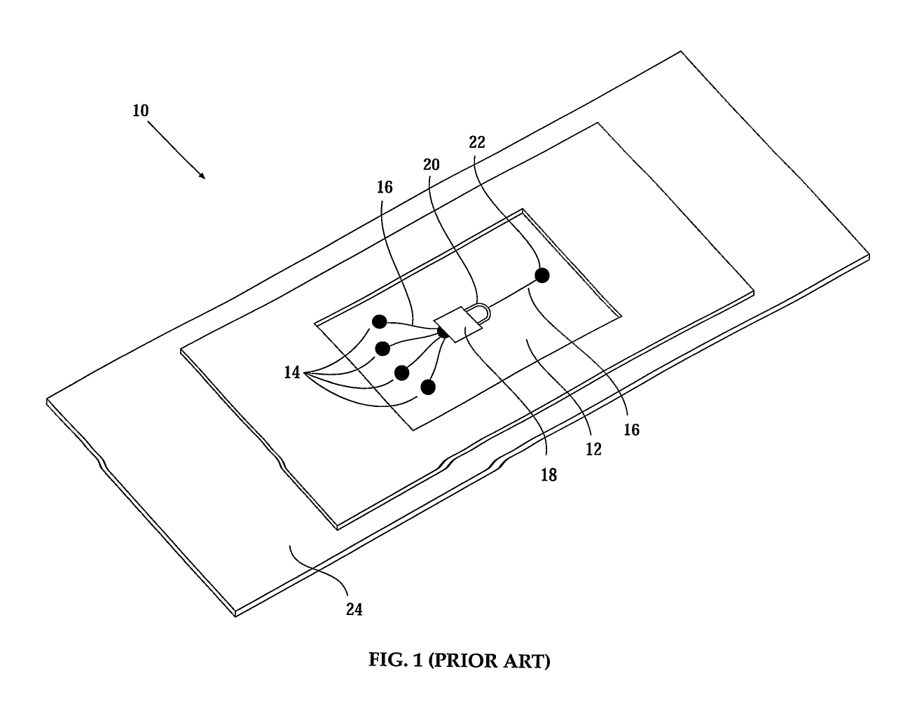

FIG. 2 (prior art) is a reproduction of a photograph of a picowell-bearing device of PCT Patent Application No. IL01/00992;

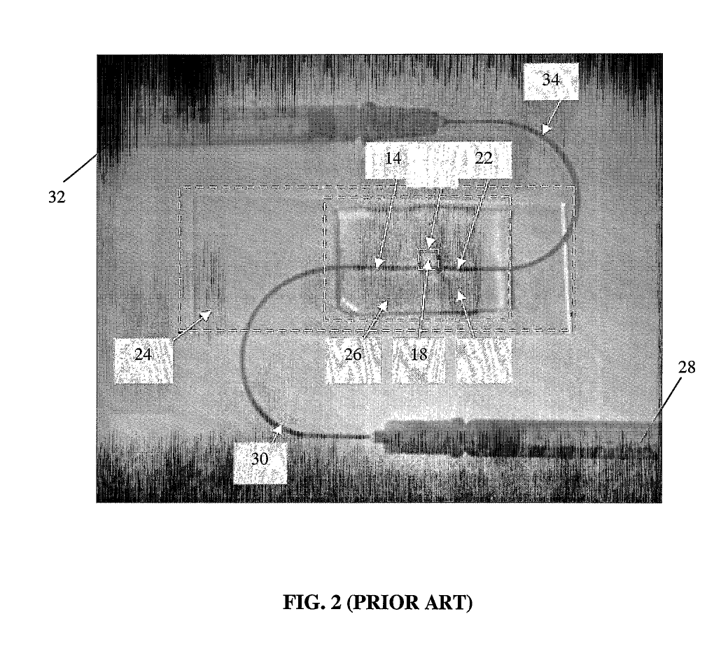

FIG. 3 (prior art) is a reproduction of a photograph of a cell-populated well-array of a carrier of a picowell-bearing device of PCT Patent Application No. IL01/00992;

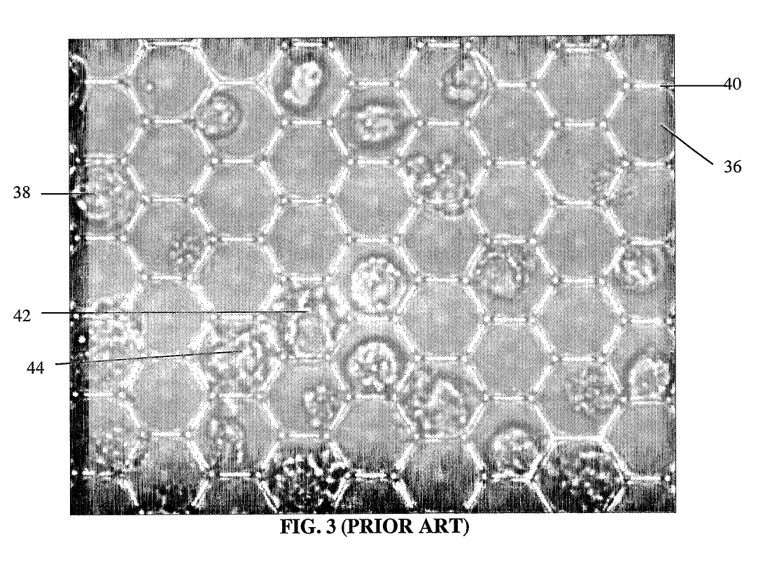

FIG. 4 is a schematic depiction of a device of the present invention with a smooth cell holder, in cross-section;

FIG. 5 is a schematic depiction of a device of the present invention with a cell holder provided with a picowell array, in cross-section;

FIG. 6 is a schematic depiction of a device of the present invention having an irradiation component disposed below the cell holder, in cross-section; and

FIG. 7 is a schematic depiction of a device of the present invention with a plurality of independently addressable radiation sources.

### PREFERRED EMBODIMENTS OF THE INVENTION

The present invention is of a method for immobilizing at least one selected cell of a group of cells by placing the group of cells in functional proximity of a radiation sensitive medium, the radiation sensitive medium having a first state that does not immobilize cells and a second state that immobilizes cells wherein the radiation sensitive medium locally transforms from the first state to the second state upon exposure to radiation. The radiation sensitive medium in proximity of the selected cell or cells is irradiated, leading to a transformation of the radiation sensitive medium from the first state to the second state, immobilizing the cell or cells. The present invention is also of a device for implementing the method of the present invention.

The principles and uses of the teachings of the present invention may be better understood with reference to the accompanying description, figures and examples. In the figures, like reference numerals refer to like parts throughout.

Before explaining at least one embodiment of the invention in detail, it is to be understood that the invention is not limited in its application to the details set forth herein. The invention can be implemented with other embodiments and can be practiced or carried out in various ways. It is also understood that the phraseology and terminology employed herein is for descriptive purpose and should not be regarded as limiting.

Generally, the nomenclature used herein and the laboratory procedures utilized in the present invention include techniques from the fields of biology, chemistry, engineering and physics. Such techniques are thoroughly explained in the literature.

Unless otherwise defined, all technical and scientific terms used herein have the same meaning as commonly understood by one of ordinary skill in the art to which the invention belongs. In addition, the descriptions, materials, methods, and examples are illustrative only and not intended to be limiting. Methods and materials similar or equivalent to those described herein can be used in the practice or testing of the present invention.

As used herein, the terms “comprising” and “including” or grammatical variants thereof are to be taken as specifying the stated features, integers, steps or components but do not preclude the addition of one or more additional features, integers, steps, components or groups thereof. This term encompasses the terms “consisting of” and “consisting essentially of”.

The phrase “consisting essentially of” or grammatical variants thereof when used herein are to be taken as specifying the stated features, integers, steps or components but do not preclude the addition of one or more additional features, integers, steps, components or groups thereof but only if the additional features, integers, steps, components or groups thereof do not materially alter the basic and novel characteristics of the claimed composition, device or method.

The term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts. Implementation of the methods of the present invention involves performing or completing selected tasks or steps manually, automatically, or a combination thereof.

Herein, the term “active entity” is understood to include chemical, biological or pharmaceutical entities including any natural or synthetic chemical or biological substance that influences a cell with which the entity is in contact. Typical active entities include but are not limited to active pharmaceutical ingredients, antibodies, antigens, biological materials, chemical materials, chromatogenic compounds, drugs, enzymes, fluorescent probes, immunogenes, probes, ligands, nucleic acids, nutrients, peptides, physiological media, proteins, receptors, selective toxins and toxins.

Herein, by “probe” is meant an active entity that upon interaction with some stimulus produces an observable effect. In the context of the present invention, by stimulus is meant, for example, a specific second active entity (such as a molecule) released by a cell. By “probe” is also meant an active entity that upon interaction with a population of cells produces a different observable effect with cells of a different type or having different attributes or characteristics. By observable effect is meant, for example, a visible effect, for example a change in color or emission of light. Typical probes include fluorescein diacetate (1 micromolar), acridine orange (5 micromolar), rhodamine 123 (1 micromolar), FDA, PI, Annexin V, anti-GlycophorinA antibody labeled with allophycocanin for the study of GFP expression.

Herein, by “picowell array” is meant a group of one or more picowells, preferably a plurality of picowells, preferably a plurality of picowells arranged in an orderly fashion.

Some embodiments of the present invention include components that are transparent or are made of a transparent material. By “transparent” is meant that the component or material is substantially transparent to at least one wavelength of light (preferably a range of wavelengths) in at least part of the visible light spectrum, the ultraviolet light spectrum, microwave radiation and/or of infrared radiation, preferably the visible light spectrum.

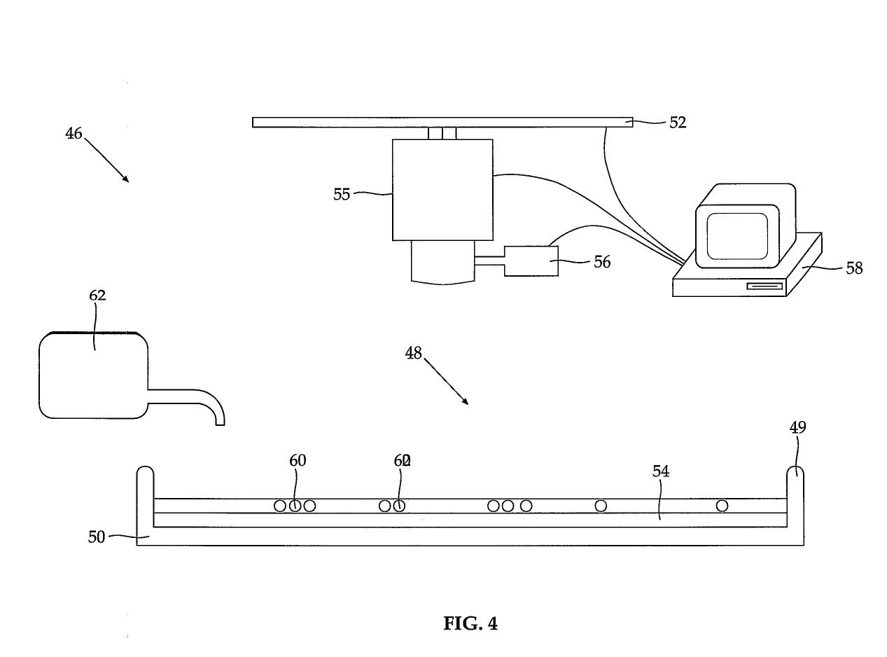

An embodiment 46 of a device of the present invention is depicted in FIG. 4. An upper surface 48 of a cell holder 50 is substantially a smooth and featureless planar area delineated by walls 49 configured to confine fluids above upper surface 48. Upper surface 48 is entirely covered with a radiation sensitive medium 54, a uniform ca. 10-micron thick layer of a relatively viscous fluid UV curable adhesive (e.g., Loctite® Product 3201 or 3211, Henkel Japan Ltd., Yokohama, Japan). Disposed above upper surface 48 is an observation component 55 (e.g., an Olympus BX61 motorized research microscope, Olympus America Inc., Melville, N.Y., USA coupled to a video display). Irradiation component 56 is an 375 nm ultraviolet laser (e.g., Lasiris™ TEC Laser, StockerYale, N.H., USA) configured to project a beam of coherent ultraviolet light coaxially with the optical axis of observation component 55. The beam of irradiation component 56 is focussed so as to form a microbeam that irradiates upper surface 48 in a 10 micron diameter circle. Irradiation component 56 is activated by control component 58. Both observation component 55 and irradiation component 56 can be moved relative to cell holder 50 in parallel to upper surface 48 using an X-Y table 52, allowing all locations of upper surface 48 to be observed using observation component 55 and irradiated using irradiation component 56. An active entity dispenser 62 is configured to dispense an active entity onto upper surface 48 of cell holder 50.

A sample including a plurality of living cells 60 (e.g., K562 Human Erythroid, MOLT-4, Human T Lymphoblasts, Peripheral Blood Lymphocytes (PBL, primary cells), Jurkat T Cell Line, Promonocyte U937) in an aqueous medium is dispensed onto upper surface 48. As upper surface 48 is planar, cells 60 settle onto upper surface 48 substantially coplanarily, resting on the layer of radiation sensitive medium 54. An operator activates active entity dispenser 62 to dispense a double-staining probe solution of FITC-Annexin V (1 μg/ml from R&D Systems (Minneapolis, Minn., USA)) and propidium iodide (2.5 μg/ml, from Sigma-Aldrich (St. Louis, Mo., USA)). As is known to one skilled in the art, in such a double-staining solution apoptopic cells are marked by a typical fluorescent signal by interaction with the Annexin V while dead cells are marked by a typical fluorescent signal by interaction with the propidium iodide. After sufficient time has passed to allow cells 60 to react with the probe solution, the operator uses X-Y table 52 to scan upper surface 48 and observe all cells 60 resting on upper surface 48. Whenever an apoptopic or dead cell 60 is observed, the operator uses control component 58 to activate irradiation component 56 to produce a microbeam of ultraviolet radiation to irradiate the vicinity of that cell 60. Being a light curable adhesive, radiation sensitive medium 54 transforms from a first fluid state to a second solid state where illuminated by the microbeam of ultraviolet radiation. As a result of the transformation, parts of the selected cells interacting with or inside radiation sensitive medium 54 are trapped in the radiation sensitive medium 54, immobilizing the selected cells.

When the entire upper surface 48 is scanned and all dead or apoptopic cells 60 have been immobilized, the non-immobilized (that is, viable) cells 60 are isolated, for example by the application of a washing solution flowing in parallel to upper surface 48. In such a way a solution of viable cells 60, devoid of dead and apoptopic cells is produced.

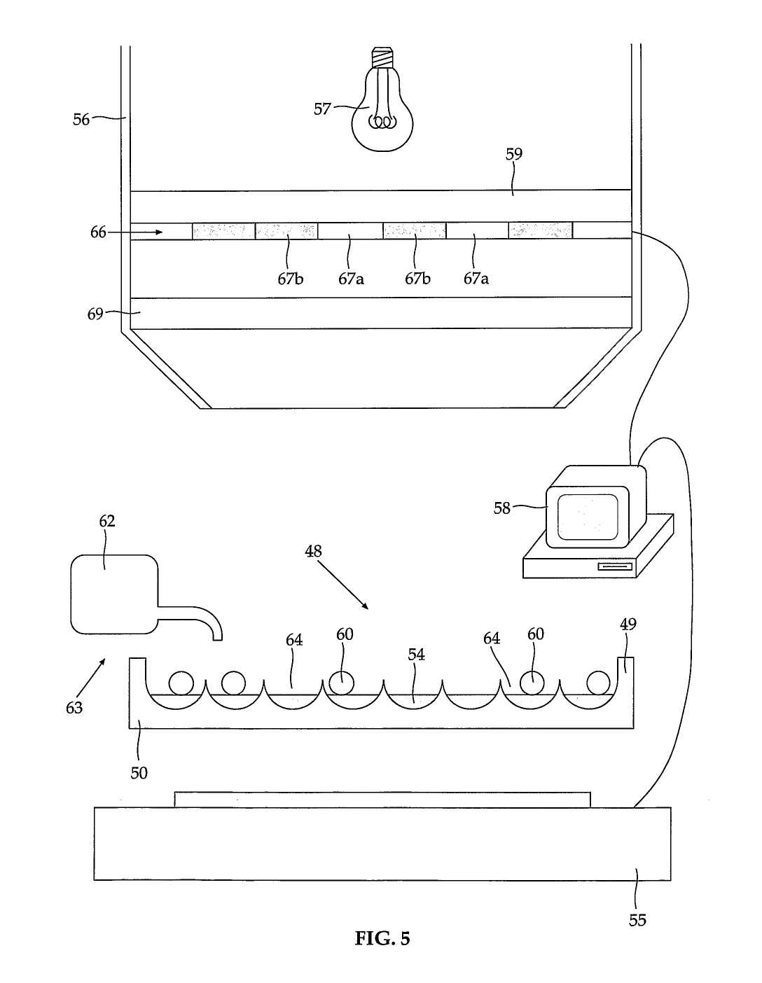

An additional embodiment 62 of a device of the present invention is depicted in FIG. 5. An upper surface 48 of a transparent cell holder 50 is entirely covered with knife-edged 10-micron picowells 64 arranged in a hexagonally packed array constituting a picowell array, analogous to picowell arrays as described, for example, in PCT Patent Application No. IL01/00992 published as WO 03/03582, in PCT Patent Application No. IL04/00571 published as WO 04/113492 and in PCT Patent Application No. IL04/00661 published as WO 05/007796, all of the Applicany. As discussed in the above references, picowells 64 are localizing features, localizing cells 60 held therein at addressable locations on upper surface 48. Deposited in the bottom of each picowell 64 is a radiation sensitive medium 54, a relatively low-viscosity fluid UV light curable adhesive (e.g., Loctite® Product 3301, Henkel Japan Ltd., Yokohama, Japan).

Disposed below upper surface 48 is an observation component 55 (e.g., an Olympus BX61 motorized research microscope, Olympus America Inc., Melville N.Y., USA coupled to a video display). Observation component 55 is configured to simultaneously observe all picowells 64.

Control component 58 is functionally associated with observation component 55 and configured to autonomously identify cells 60 and to autonomously designate cells 60 as being selected for immobilization based on input from observation component 55, for example, in accordance with the teachings of unpublished copending PCT Patent Application No. IL05/000719 of the Applicant. Irradiation component 56 disposed above upper surface 48 comprises a radiation source 57 (a medium-pressure mercury lamp), a collimator 59 and an adjustable mask 66. Control component 58 is functionally associated, inter alia, with adjustable mask 66. Adjustable mask 66 is a substantially planar component comprising a rectangular grid of liquid crystal pixels 67 that can be independently rendered opaque (67b) or transparent (67a). When radiation source 57 is activated, light produced thereby passes through collimator 59, through transparent liquid crystal pixels 67a and through lens 69, producing a light and dark image of the transparent and opaque liquid crystal pixels 67a and 67b. Adjustable mask 66 and lens 69 are configured so that there exits at least one pixel that corresponds to every picowell 64, that is that every picowell 64 can be illuminated by rendering at least one liquid crystal pixel 67 transparent.

An active entity dispenser 62 is configured to dispense an active entity onto upper surface 48 of cell holder 50.

A sample including a plurality of living cells 60 (e.g., K562 Human Erythroid, MOLT-4, Human T Lymphoblasts, Peripheral Blood Lymphocytes (PBL, primary cells), Jurkat T Cell Line, Promonocyte U937) in an aqueous medium is dispensed onto upper surface 48. Cells 60 settle by sedimentation, each cell 60 into an individual picowell 64 to rest on radiation sensitive medium 54. An operator activates active entity dispenser 62 to dispense a double-staining solution of FITC-Annexin V (1 μg/ml from R&D Systems (Minneapolis, Minn., USA)) and propidium iodide (2.5 μg/ml, from Sigma-Aldrich (St. Louis, Mo., USA)). After sufficient time has passed to allow cells 60 to react with the probe, observation component 55 is activated to acquire an image of cells 60 held in picowells 64 on upper surface 48.

In accordance with the teachings of unpublished copending PCT Patent Application No. IL05/000719 of the Applicant, control component 58 autonomously identifies each cell 60 and determines according to predetermined criteria which cells 60 are dead or apoptopic and therefore are selected for immobilization and which cells 60 are viable. Pixels 67a of adjustable mask 66 associated with picowells 64 holding dead or apoptopic cells 60 to be immobilized are set by control component 58 to be transparent. Pixels 67b of adjustable mask 66 associated with picowells 64 holding viable cells 60 are set by control component 58 to be opaque. Radiation source 57 is activated to project light onto adjustable mask 66. Light passing through transparent pixels 67a transforms radiation sensitive medium 54 in associated picowells 64 from a first fluid state to a second solid state. As a result of the transformation, dead or apoptopic cells 60 held in such picowells 64 are trapped in the radiation sensitive medium 54, immobilizing those cells 60.

Subsequent to irradiation the non-immobilized cells are isolated, for example by the application of a washing solution flowing in parallel to upper surface 48. In such a way a solution of viable cells 60, devoid of dead and apoptopic cells is produced.

In an alternate embodiment, adjustable mask 66 is provided with 10 micron square liquid crystal pixels 67, each such pixel associated with a picowell 64 so that when a liquid crystal pixel 67 is transparent projected light passes through pixel 67 to impinge on a respective picowell 64. Such an embodiment works substantially the same as discussed above with a substantial exception that there is no need for a lens 69.

In an alternate embodiment, irradiation component 56 comprises a digitally controlled micromirror array functionally associated with control component 58 allowing reflection of radiation from a radiation source 57 only onto desired picowells, e.g., using Digital Light Processing technology™ (Texas Instruments DLP Products, Plano, Tex., USA), see for example, U.S. Pat. No. 6,775,174, U.S. Pat. No. 6,775,174, U.S. Pat. No. 5,600,383 and U.S. Pat. No. 5,583,688)

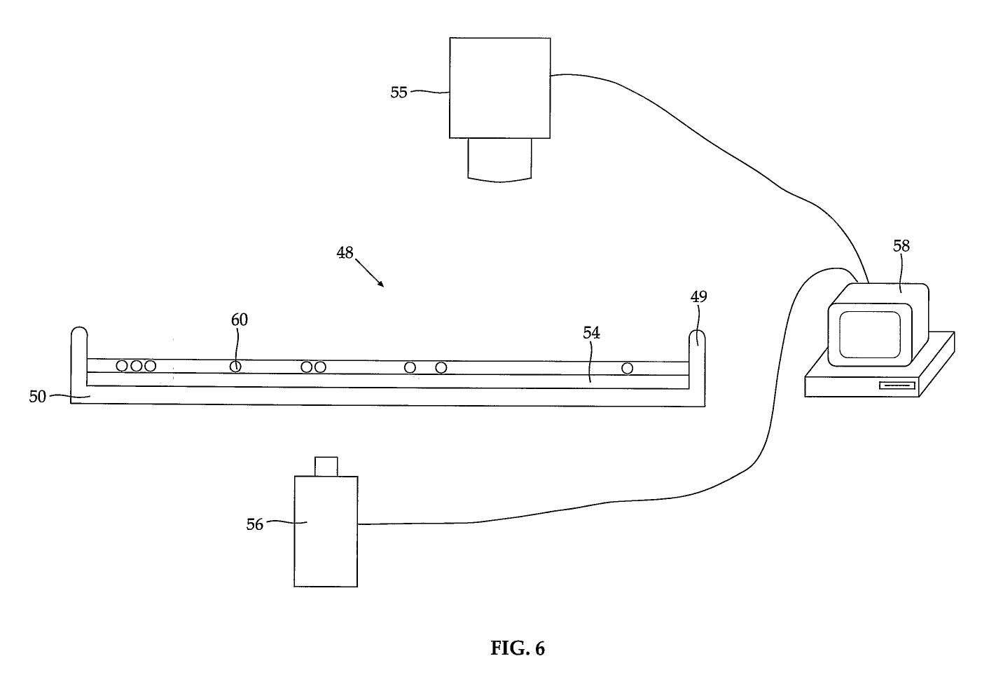

In an embodiment of the present invention depicted in FIG. 6, an irradiation component 56 is configured to irradiate a radiation sensitive medium 54 from below upper surface 48. As in such embodiments the radiation must pass through cell holder 50, cell holder 50 must be transparent to the frequencies of radiation produced by irradiation component 56 required to transform radiation sensitive medium 54 from the respective first state to the respective second state.

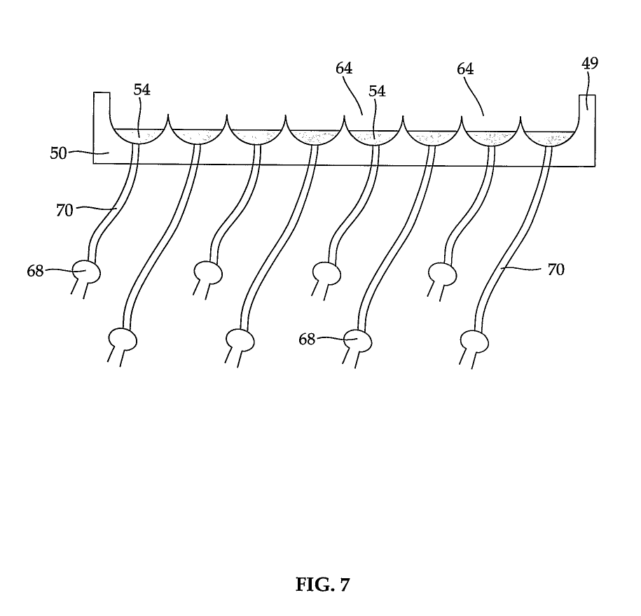

In an embodiment of the present invention depicted in FIG. 7, an irradiation component 56 comprises a plurality of independently addressable radiation sources 68, each such independently addressable radiation source associated with a specific picowell 64 through an optical fiber 70. Irradiation of radiation sensitive medium 54 in a given picowell 64 is performed independently of irradiation of other picowells, simultaneously or sequentially. In an alternative embodiment, each picowell is associated with at least one independently addressable light-emitting elements of an AMEL (Active Matrix Electroluminesence) device (Planar Systems, Inc., Beaverton, Oreg., USA).

In the above embodiments, a cell holder 50 is configured so that cells 60 settle to rest on upper surface 48 substantially coplanarily. Although not necessary for implementing the teachings of the present invention, coplanarity is preferred for ease of optical study and observation of cells 60: coplanarity allows for optical observation of many cells (whether by scanning or simultaneously using a wide-angle observation component) without the need for time consuming and technically difficult to implement refocusing.

In general, the frequencies of radiation required to transform a given radiation sensitive medium 54 from a respective first state to a respective second state depend on the nature of the radiation sensitive medium 54 and include frequencies of visible light, infrared radiation, ultraviolet radiation and microwave radiation.

In embodiments of the present invention, cells 60 are found entirely within a layer of radiation sensitive medium 54 when in the first state. A disadvantage of such an embodiment is that cells 60 are then found in a potentially non-natural environment that may affect cell behavior. In preferred embodiments, at least part of cells 60 are not found within the radiation sensitive medium 54 in the first state (e.g., the top part of the cells emerges above a layer of radiation sensitive medium 54) or even cells 60 are substantially not found within radiation sensitive medium 54 but rather simply rest on top of the radiation sensitive medium 54 in the first state.

In embodiments of the present invention the radiation sensitive medium 54 is more viscous in the second state (e.g., a solid or a gel) than in the first state (e.g., a liquid). Generally in such embodiments, a selected cell 60 rests on the surface of the radiation sensitive medium 54 with parts of selected cell 60 interacting with or inside radiation sensitive medium 54. The irradiation transforms the radiation sensitive medium 54 to the second state to immobilize cell 60.

In some embodiments, the radiation sensitive medium 54 is fluid in the first state and solid in the second state. Typical examples of such radiation sensitive media are light-curable adhesives such as light curable cyanoacrylates, light curable acrylics, light curable epoxies and light curable silicones. Suitable light curable adhesives are available, inter alia, from Henkel Japan Ltd., Loctite Division (Yokohama, Japan), Star Technology, Inc., (Waterloo Ind., USA), Grace Specialty Polymers (Lexington Mass., USA) and Dymax Corporation (Rocky Hill Conn., USA).

In some embodiments, the radiation sensitive medium 54 is fluid in the first state and gel in the second state. A typical example of such radiation sensitive media are free-radical induced gelatinating polyethylene glycols, see for example, Koh et al. in Langmuir 2002, 18, 2459-2462 for technical details.

In embodiments of the present invention the radiation sensitive medium 54 is more viscous in the first state (e.g., a solid or a gel) than in the second state (e.g., a liquid). Generally in such embodiments, a selected cell 60 rests on the surface of the radiation sensitive medium 54 until radiation sensitive medium 54 is irradiated. The irradiation transforms radiation sensitive medium 54 to the second state into which the selected cell 60, in whole or partially, sinks. When irradiation is stopped, radiation sensitive medium 54 reverts to the first state or to another state that is sufficiently viscous to immobilize cell 60.

In some embodiments, the medium is gel in the first state and a fluid in the second state. A typical such example is modified collagen gel solutions (see for example, De Vore et al. in J. Refract. Surg. 1995; 11:50-55; Stevens et al. in Cornea 1999; 18:155-63; Pallikaris et al. in Seminars in Opthalmology. 2000; 15:160-169 and Kremer et al. in Cornea. 2002; 21:28-32).

In some embodiments, the radiation sensitive medium 54 is a solid in the first state and a fluid in the second state. Typical such examples are waxes and paraffins. When waxes and the like are used, it is generally preferably to tale care to only soften rather than melt radiation sensitive medium 54 completely as waxes are often less dense than the aqueous medium in which cells 60 are found so complete melting leads to wax floating away. Alternatively, a high-density wax such as a chlorinated wax (available, for example, from Sawmatic, Shropshire, United Kingdom) is used as the radiation sensitive medium 54.

In some embodiments of the present invention, a radiation sensitive medium 54 comprises chemical functional groups (generally bound to upper surface 48) configured to bind to cells 60 and in such a way immobilize cells 60. Preferably, in the first state of such a radiation sensitive medium 54 the chemical functional groups are in a nonreactive inactivate state, for example are associated with radiation labile protecting groups and in the second state the chemical functional groups are in a reactive state induced by exposure to radiation. The number of suitable chemistries for implementing such an embodiment of the present invention is very high so cannot be rigorously discussed herein. Representative suitable functional groups include commercially available photoreactive crosslinking reagents from Invitrogen Corporation (Carlsbad Calif., USA). An exemplary radiation sensitive medium 54 having chemical functional groups to immobilize cells 60 is based on N-(2-pyridyldithio) ethyl-4-azidosalicylamide (PEAS:AET) commercially available from Invitrogen Corporation (Carlsbad Calif., USA). To form the layer of radiation sensitive medium 54, glutathione (Pierce, Rockford, Ill. USA) is immobilized on upper surface 48 of cell holder 50. The N-(2-pyridyldithio) ethyl part of the PEAS:AET undergoes a disulfide-thiol interchange, leading to attachment of the ethyl-4-azidosalicylamide residue of the PEAS:AET to upper surface 48. When the ethyl-4-azidosalicylamide residue is irradiated, a nitrogen radical is formed and reacts with proteins on the surface of a cell 60 in the proximity of the radical, thereby immobilizing cell 60.

In some embodiments, the process of immobilizing a cell 60 with a radiation sensitive medium 54 may damage or kill cell 60. In embodiments of the present invention that are directed to isolating desired cells by immobilizing undesired cells, this is not generally a concern. An advantage of the present invention is that cells 60 that are killed or damaged are immobilized so do not contaminate the isolated desired cells.

Described hereinabove is that cells 60 are exposed to a single stimulus (a probe) in order to differentiate between two types of cell 60 in order to select which cells 60 to immobilize. Upon perusal of the description herein one skilled in the art is able to combine the teachings of the present invention with devices and methods known in the art including those disclosed in unpublished pending U.S. patent application Ser. No. 10/276,080, PCT Patent Application No. IL01/00992 published as WO 03/035824, PCT Patent Application No. IL04/000194 published as WO 04/077009, PCT Patent Application No. IL04/000571 published as WO 04/113492, PCT Patent Application No. IL04/000661 published as WO 05/007796, unpublished PCT Patent Application No. IL05/000719 and unpublished PCT Patent Application No. IL05/000801, all of the Applicant, to perform more complex experiments involving the exposure of cells 60 to more than one active entitity or stimulus. For example, cells 60 held in a cell holder 50 may be undergo exposure to two or more stimuli, such as two or more active entities, especially such active entities as drug candidates, selective toxins, active pharmaceutical ingredients and the like.

Described above is an embodiment of a cell holder 50 that is featureless. That said, in preferred embodiments, a cell holder 50 comprises localizing features such as picowells, preferably arrayed in a picowell array. Preferably picowells of a picowell array are juxtaposed. By juxtaposed is meant that in an area where picowells are found, most of the area is picowell area and little of the area is inter picowell area. Such a configuration allows simple high-density loading (by sedimentation) of cells 60 in well-defined locations with no formation of cell aggregates. According to a feature of the present invention, by juxtaposed is meant that the inter picowell area between two picowells is less than or equal to 0.35, 0.25, 0.15, 0.10 or even 0.06 of the sum of the areas of the two picowells. In certain embodiments of the present invention it is preferred that the inter picowell area be substantially zero, that is that the rims of picowells are substantially knife-edged.

It is generally preferred that the picowells be small so as to avoid having a large number of cells held in any one picowell. Thus, generally, the dimensions of the picowells are generally up to about 200, 100, 50, 25, 20 or even 10 microns. By dimensions is meant the usual meaning of the word and is dependent on the shape of the picowell. For example, for hexagonal or circular picowells, the term dimension refers to diameter. For square or triangular picowells is meant the longest dimension of the square or triangle, respectively. The exact dimensions of individual picowells depends on the type (and consequently size) of cells to be studied and the types of experiments and studies that are to be performed. Since different types of cells have different sizes, generally a picowell array of the present invention has picowells of a size to accommodate one or more cells of the type to be studied. In some embodiments it is preferred that an individual picowell be of a size so as to hold no more than one living cell of a certain size.

In some embodiments of the present invention, picowells are dimples or depressions on the bottom surface of the picowell array. In other embodiments the picowells are substantially enclosures of dimensions so that at least one cell of a certain size is containable, preferably but not necessarily substantially entirely, within the enclosure, each enclosure having an opening at the surface, the opening defined by a first cross section of a size allowing passage of cell of the certain size. The exact dimensions of the individual enclosures depends on the type (and consequently size) of cells to be studied and the types of experiments and studies that are to be performed. The volume of such enclosure picowells is typically less than 1×10⁻¹¹ liter (corresponding to the volume of a 200 micron cube), less than 1×10⁻¹² liter (corresponding to the volume of a 100 micron cube), less than 1×10⁻¹³ liter (corresponding to the volume of a 50 micron cube), less than 1×10⁻¹⁴ liter (corresponding to the volume of a 25 micron cube) and even less than 1×10⁻¹⁵ liter (corresponding to the volume of a 10 micron cube). The area of the first cross section, corresponding to the size of the opening of a respective enclosure is typically less than about 40000 micron² (corresponding to the area of a 200 micron square), 10000 micron² (corresponding to the area of a 100 micron square), 2500 micron² (corresponding to the area of a 50 micron square), 625 micron² (corresponding to the area of a 25 micron square) or even less than about 100 micron² (corresponding to the area of a 10 micron square).

Preparation of the Radiation Sensitive Medium

In general, manufacture and assembly of a device of the present invention is well within the ability of one skilled in the art upon perusal of the description and figures herein using any suitable method with which one skilled in the art is well acquainted, and when necessary, by perusing the references cited herein.

An important aspect of implementing the teachings of the present invention is the deposition of a radiation sensitive medium 54 on a cell holder 50. As discussed above and as is clear to one skilled in the art, as there are many different materials that are suitable for use as radiation sensitive media 54 for implementing the teachings of the present invention, it is not possible to provide an exhaustive discussion of how each and every suitable radiation sensitive media 54 is deposited on a given cell holder 50. The impossibility of such a discussion is exceptionally true of radiation sensitive media 54 that consist of chemical entities bound to the upper layer of the cell holder as described above. In such cases one skilled in the art is able to use well-known techniques to prepare the radiation sensitive medium 54.

That said, many a suitable radiation sensitive medium 54 is liquid in the first state. Such media are conveniently applied by pouring into a cell holder 50. To achieve a radiation sensitive medium layer having a uniform thickness it is often advantageous to use spin coating methods. An alternative method especially suitable for application of radiation sensitive media 54 to a cell holder 50 including picowells is by spraying, for example using an electrospray technique with which one skilled in the art is well acquainted. Alternatively, pouring a suspension or a solution of a radiation sensitive medium 54 in a volatile solvent also provides a uniform distribution of radiation sensitive medium 54.

In embodiments of the present invention, many suitable radiation sensitive media 54 are not fluid, e.g., are gels or solids in the first state. In such cases, it is often possible to apply the radiation sensitive medium 54 as a liquid (for example, by heating, by dissolving in a suitable solvent, or by applying the radiation sensitive medium 54 as a liquid precursor that is subsequently induced to solidify or to gel) for application as described above.

In embodiments of the present invention where the radiation sensitive media 54 is not fluid in the first state, for example when the radiation sensitive medium 54 has a wax-like consistency, it is often advantageous to place the radiation sensitive medium 54 in a cell holder 50 and to form picowells thereupon. Methods of formation of picowells on such a radiation sensitive medium 54 are known to one skilled in the art upon perusal of the description herein. A preferred method is analogous to the methods disclosed by the applicant in PCT Patent Application No. IL01/00992 published as WO 03/03582, in PCT Patent Application No. IL04/00571 published as WO 04/113492 and in PCT Patent Application No. IL04/00661 published as WO 05/007796. Such a method includes a) contacting a precursor material (e.g., molten radiation sensitive medium 54) with a template including a negative of the picowells; and b) fixing the picowells in the precursor material. Depending on the precursor material, fixing includes, but is not limited to, methods such as heating the precursor material, cooling the precursor material, curing the precursor material, polymerizing the precursor material, cross-linking the precursor material, irradiating the precursor material, illuminating the precursor material, gelling the precursor material, exposing the precursor material to a fixative and waiting a period of time. By fixative is meant an agent that causes the precursor material to change to the fixed state and is used herein as a general term for such materials as fixatives, hardeners, polymerization/crosslinking/curing initiators, catalysts and agents. It is important to note that in some cases a precursor material is produced by mixing two or more components which thereafter change to a fixed state, for example, by simply waiting a period of time. In embodiments of the present invention, the precursor material is a irreversibly deformable precursor material. Herein by irreversibly deformable precursor material is meant a material that does not recover a shape after deformation and so there is usually no need for a separate action to fix the features in the precursor material beyond separating the produced component from the template. In such cases, the precursor material does not substantially chemically change subsequent to contact with the template. Examples of suitable irreversibly deformable precursor materials include waxes, paraffins, plastics, polymers and the like. In such an embodiment, a preferred template is a stamp, and the contacting of the template with the precursor material is substantially stamping the features of the component into the precursor material, preferably under controlled thermal conditions.

In another preferred embodiment of the present invention, the precursor material is a reversibly deformable precursor material. Herein by reversibly deformable precursor material is meant a material that is capable of recovering shape after deformation and includes gellable fluids, polymerizable materials, powders, fluids and thermoplastic materials.

In a preferred embodiment, the reversibly deformable precursor material is a thermoplastic material at a pliable temperature. Subsequent to the contacting of the template but before the contact is finished, the thermoplastic material is cooled, thus fixing the desired features in the incipient component.

In another preferred embodiment, the reversibly deformable precursor material is a polymerizable material (e.g., a monomer solution, a crosslinkable polymer, a vulcanizable polymers, a polymerizable fluids or a thermosetting resin). Subsequent to the contacting of the template but before the contact is finished, the polymerizable material is polymerized, thus fixing the desired features in the incipient component. In such cases, the precursor material and the material from which the component is made are chemically dissimilar (for example, have the relationship of monomer to polymer).

Another preferred reversibly deformable precursor material is a gellable fluid. After the gellable fluid is brought in contact with the template, the features are fixed by gelling the gellable fluid to yield a gel. Gellable fluids known in the art include fluids that gel upon heating, fluids that gel upon cooling, fluids that gel upon irradiation or illumination, fluids that gel as a result of contact with a gelling reagent and fluids that gel after a period of time.

The template having a negative of the features is, for example, a stamp or a mold, and is generally made of any suitable material that is more rigid than a respective precursor material. Suitable materials include but are not limited to reversibly deformable materials, irreversibly deformable materials, ceramics, epoxies, glasses, glass-ceramics, metals, plastics, polycarbonates, polydimethylsiloxane, polyethylenterephtalate glycol, polymers, polymethyl methacrylate, paraffins, polystyrene, polyurethanes, polyvinyl chloride, silicon, silicon oxide, silicon rubbers and wax. The template is made, for example, using methods with which one skilled in the art is acquainted such as casting, embossing, etching, free-form manufacture, injection-molding, microetching, micromachining, microplating, molding, lithography or photo-lithography.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination. Although the invention has been described in conjunction with specific embodiments thereof, it is evident that many alternatives, modifications and variations will be apparent to those skilled in the art. Accordingly, the present invention is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In case of conflict, the specification herein, including definitions, will control. Citation or identification of any reference in this application shall not be construed as an admission that such reference is available as prior art to the present invention.

1-73. (canceled)

74. A method of immobilizing at least one cell from a plurality of cells comprising:
(a) placing the plurality of cells in functional proximity of a radiation sensitive medium, said radiation sensitive medium having a first state that does not immobilize cells and a second state that immobilizes cells wherein said radiation sensitive medium locally transforms from said first state to said second state upon exposure to radiation;
(b) selecting at least one cell to be immobilized; and
(c) using radiation produced by a radiation source to irradiate said radiation sensitive medium in said first state in the vicinity of said at least one selected cell so as to transform said radiation sensitive medium only in said vicinity of said at least one selected cell to said second state so as to immobilize said at least one selected cell.

75. The method of claim 74, further comprising:
(d) subsequent to said irradiation, isolating non-immobilized cells.

76. The method of claim 74, wherein said selecting said at least one cell to be immobilized includes:
(i) individually observing a cell from the group of cells; and
(ii) designating said observed cell as said selected cell wherein said designation is contingent on observing a specific property of said observed cell.

77. The method of claim 76, further comprising: prior to said irradiating,
(f) exposing said group of cells to a stimulus that induces an observable effect indicative of said specific property.

78. The method of claim 76, wherein said selected cell is irradiated prior to observing of a different cell of the group of cells.

79. The method of claim 76, wherein a plurality of cells are designated as selected cells prior to said irradiation.

80. The method of claim 74, wherein said individual cells are individually addressable.

81. The method of claim 80, wherein said individual cells are held in wells.

82. The method of claim 80, wherein said individually addressable cells are associated with individually addressable radiation sources to irradiate said radiation sensitive medium.

83. The method of claim 74, wherein, during said irradiation a single radiation source is used to sequentially irradiate the vicinity of a plurality of selected cells.

84. The method of claim 74, wherein, during said irradiation a single radiation source is used to simultaneously irradiate the vicinity of a plurality of selected cells.

85. The method of claim 74, wherein cells of said plurality of cells are not immersed in said radiation sensitive medium in said first state.

86. The method of claim 74, wherein cells of said plurality of cells rest on top of a layer of said radiation sensitive medium in said first state.

87. The method of claim 74, wherein said radiation sensitive medium is more viscous in said first state than in said second state.

88. The method of claim 74, wherein said radiation sensitive medium comprises chemical functional groups configured to bind to cells held in said cell holder, wherein in said first state said chemical functional groups are non-reactive and transformation of said radiation sensitive medium to said second state includes radiation-induced activation of the chemical functional groups.

89. The method of claim 74, wherein said radiation source comprises a laser.

90. The method of claim 74, wherein said radiation is in the form of a beam having a diameter of less than 50 micron.

91. A device for immobilizing cells comprising:
(a) a cell holder for holding cells having an upper surface;
(b) on said upper surface a radiation sensitive medium, said radiation sensitive medium having a first state that does not immobilize cells held in said cell holder and a second state that immobilizes cells held in said cell holder wherein said radiation sensitive medium locally transforms from said first state to said second state upon exposure to radiation;
(c) an observation component configured to allow observation of individual cells held in said cell holder;
(d) an irradiation component configured to selectively irradiate said radiation sensitive medium in proximity of selected cells held in said cell holder with radiation; and
(e) a control component functionally associated with said irradiation component, configured to direct said irradiation component to irradiate said radiation sensitive medium in proximity of selected cells held in said cell holder.

92. The device of claim 91, wherein said control component is functionally associated with said observation component and configured to autonomously designate cells held in said cell holder as selected cells based on input of said observation component.

93. The device of claim 91, wherein said irradiation component is configured to irradiate said radiation sensitive medium in proximity of a single cell of a plurality of cells held in said cell holder at any one time.

94. The device of claim 91, wherein said irradiation component is configured to project a beam of radiation.

95. The device of claim 94, wherein said beam of radiation has a diameter of less than 50 micron.

96. The device of claim 91, wherein said irradiation component is configured to simultaneously irradiate said radiation sensitive medium in proximity of a plurality of selected cells from a plurality of cells held in said cell holder at any one time.

97. The device of claim 91, wherein said irradiation component comprises a plurality of independently addressable radiation sources.

98. The device of claim 91, wherein said control component comprises an adjustable mask.

99. The device of claim 91, wherein in said first state, said radiation sensitive medium is configured to only partially surround cells held cells held in said cell holder so that at least part of a said surrounded cell emerges from said radiation sensitive medium.

100. The device of claim 74, wherein in said first state, said radiation sensitive medium is configured to allow cells held in said cell holder to rest thereupon.

101. The device of claim 91, wherein said radiation sensitive medium is more viscous in said second state than in said first state.

102. The device of claim 91, wherein said radiation sensitive medium is more viscous in said first state than in said second state.

103. The device of claim 91, wherein said radiation sensitive medium comprises chemical functional groups configured to bind to cells held in said cell holder, wherein in said first state said chemical functional groups are non-reactive and transformation of said radiation sensitive medium to said second state includes radiation-induced activation of the chemical functional groups.

104. The device of claim 91, further comprising localizing features on said upper surface configured to localize cells held in said cell holder at specific locations of said upper surface.
